(19) United States
(12) Patent Application Publication
(10) Pub. No.: US 2013/0089530 A1
(43) Pub. Date: Apr. 11, 2013

(54) METHODS AND COMPOSITIONS FOR TREATING PARASITIC WORM INFECTIONS IN A MAMMAL

(71) Applicant: MICROBES, INC., The Woodlands, TX (US)

(72) Inventor: Marc J. Rodriguez, The Woodlands, TX (US)

(73) Assignee: MICROBES, INC., The Woodlands, TX (US)

(21) Appl. No.: 13/649,602

(22) Filed: Oct. 11, 2012

Related U.S. Application Data
(60) Provisional application No. 61/545,710, filed on Oct. 11, 2011.

(51) Int. Cl. A61K 35/74; A61P 33/00

(52) U.S. Cl. 424/93.41; 424/93.4; 424/195.16; 424/93.46; 424/93.48; 424/93.45; 424/93.47; 424/93.42; 424/93.44; 424/93.43

(57) ABSTRACT

The invention relates to compositions for treating parasitic worm infections in a mammal. The composition includes an amount of intracellular components of lysed, beneficial, soil-inhabiting yeast cells sufficient to treat parasitic worm infections in a mammal; and/or an amount of whole or lysed soil-inhabiting bacteria cells, wherein the amount is sufficient to treat parasitic worm infections in a mammal, and optionally a pharmaceutically suitable carrier.

### CROSS-REFERENCE TO RELATED APPLICATION

This application claims the benefit of U.S. Provisional Application No. 61/545,710 filed Oct. 11, 2011, which is incorporated herein by reference in its entirety.

### BACKGROUND OF THE INVENTION

The purpose of this invention is to provide a unique product that has reduced toxicity, and that will effectively treat parasitic worm infections in a mammal.

Intestinal parasites are micro-organisms that live in the intestines of mammals. Infection by intestinal parasitic worms is widespread throughout the world, affecting hundreds of millions of people and mammals.

A parasite survives by living off of the host it infects, robbing the host of nutrients and, leaving behind toxic waste. One of the most common kinds of parasitic worms is the roundworm (also known as nematodes). Roundworms live in the intestines of the infected mammal and their numbers build up through repeated infection. It is possible to be infected with more than one kind of worm.

Roundworms can release tens of thousands of eggs at a time and it's the eggs or the freshly hatched larvae that are inadvertently picked up from walking barefoot or gardening in infected soil. Parasitic worms may also spread through contaminated water and feed.

Common pets such as dogs and cats are highly susceptible to parasitic nematode infection through mosquito bites and other vector transmissions. For example, Dirofilaria spp (dog heartworm), is a major cause of dog mortalities every year. Additionally, small and large animal ruminants (e.g. goats, sheep and cattle) are frequently infected by eating grass carrying the nematode larvae, and thus become carriers of parasitic nematodes. This is a major cause for concern, especially in developing countries where prevention and control methods are limited. In addition, parasitic worms can be transferred from pet to owner.

Parasitic worms are responsible for many health conditions including diarrhea, gastrointestinal upset, vaginal irritation, joint pain, nervous diseases, immune dysfunction and chronic fatigue. Long term, undetected infection can cause many systemic problems. For the very old, very young or immunocompromised, a parasitic infection can be extremely problematic.

Presently, three drug classes are used in the treatment of parasitic nematode infections in mammals. However, clinical research and studies conducted primarily in third world countries has demonstrated a growing resistance to all three drug classes. The first class consists of the Benzimidazoles (e.g. fenbendazole, albendazole), the second class consists of nicotinics (e.g. levamisole, morantel and pyrantel) and the third class comprises macrolides (e.g. avermectin, ivermectin, doramectin and moxidectin).

There is a major cause for concern over the growing genetic resistance markers spreading throughout nematode populations. There exists a strong demand for novel, natural treatments for parasitic worm infections that are safe, non-invasive and effective.

### SUMMARY OF THE INVENTION

In a first aspect, the invention relates to a composition for treating parasitic worm infections in a mammal. The composition comprises an amount of intracellular components of lysed, soil inhabiting yeast cells that are beneficial to plants and optionally a pharmaceutically suitable carrier. The parasitic worm can be a cestode, nematode or trematode, or any combination thereof. The composition can also include amino acids. Additionally, the composition can further comprise an amount of whole or lysed bacteria cells, and optionally a pharmaceutically suitable carrier, wherein the bacteria cells are from soil inhabiting bacteria that are beneficial to plants.

In a second aspect, the invention relates to a method for treating parasitic worm infections in a mammal. The method comprises administering to the mammal a composition comprising an amount of intracellular components of lysed, soil inhabiting yeast cells that are beneficial to plants and optionally a pharmaceutically suitable carrier. The parasitic worm can be a cestode, nematode or trematode, or any combination thereof. The composition can also include amino acids. Additionally, the composition can further comprise an amount of whole or lysed bacteria cells, and optionally a pharmaceutically suitable carrier, wherein the bacteria cells are from soil inhabiting bacteria that are beneficial to plants.

In a third aspect, the invention relates to a composition for treating parasitic worm infections in a mammal. The composition comprises an amount of whole or lysed bacteria cells, and optionally a pharmaceutically suitable carrier, wherein the bacteria cells are from soil inhabiting bacteria that are beneficial to plants. The parasitic worm can be a cestode, nematode or trematode, or any combination thereof. The composition can also include amino acids.

In a fourth aspect, the invention relates to a method for treating parasitic worm infections in a mammal. The method comprises administering to the mammal a composition comprising an amount of whole or lysed bacteria cells, and a pharmaceutically suitable carrier. The bacteria cells are from soil inhabiting bacteria that are beneficial to plants. The parasitic worm can be a cestode, nematode or trematode, or any combination thereof. The composition can also include amino acids.

In a fifth aspect, the invention relates to a composition for treating parasitic worm infections in a mammal. The composition comprises:
(a) an amount of intracellular components of lysed, soil-inhabiting yeast cells that are beneficial to plants sufficient to treat parasitic worm infections in the mammal;
(b) an amount of whole or lysed bacteria cells, and a pharmaceutically suitable carrier, wherein the bacteria cells are from soil inhabiting bacteria that are beneficial to plants sufficient to treat parasitic worm infections in the mammal; and
(c) optionally a pharmaceutically suitable carrier.

In a sixth aspect, the invention relates to a method for treating parasitic worm infections in a mammal. The method comprises administering to the mammal a composition comprising:
(a) an amount of intracellular components of lysed, soil-inhabiting yeast cells that are beneficial to plants sufficient to treat parasitic worm infections in the mammal;
(b) an amount of whole or lysed bacteria cells, and a pharmaceutically suitable carrier, in an amount is sufficient to treat parasitic worm infections in the mammal; and
(c) optionally a pharmaceutically suitable carrier.

### DETAILED DESCRIPTION OF THE INVENTION

### Parasitic Worm Infection

According to the invention, parasitic worm infection is any infection that is caused by a parasitic worm. The parasitic worm can be a nematode, cestode or trematode, or combination thereof. Some examples of specific worms include roundworm, pinworm, threadworm, flukeworm, tapeworm, whipworm or hookworm. In a preferred embodiment, the worm is a roundworm.

The parasitic worm infection can present with or without symptoms in the host.

### Soil-Inhabiting Microorganisms

The rhizosphere is the soil surrounding the roots of a plant in which complex relations exist between the soil, plants with roots in the soil, and microorganisms that inhabit the soil. The roots influence the chemistry and biology of the rhizosphere, including pH and nitrogen transformations. The microorganisms affect the plants whose roots are in the rhizosphere.

In this specification, soil-inhabiting microorganisms include microorganisms that colonize, and/or live in the soil and/or the rhizosphere of, a plant, and that have a significant and positive effect on the plant. Such microorganisms include, for example, yeast cells and bacteria cells. Examples of such bacteria include rhizobacteria cells and rhizosphere-associated bacteria cells. Rhizobacteria are a category of bacteria that colonize plant roots (e.g., Rhizobium). Rhizosphere-associated bacteria do not colonize plant roots, but live in the perimeter of the roots in the rhizosphere (e.g., Bacillus).

### Compositions

The compositions of the invention may or may not comprise a suitable carrier, preferably pharmaceutically suitable carrier. Suitable carriers are well known in the art. In this specification, a pharmaceutical carrier is considered synonymous with a vehicle or an excipient as understood by practitioners in the art. Examples of carriers include starch, milk, sugar, certain types of clay, gelatin, stearic acid or salts thereof, magnesium or calcium stearate, talc, vegetable fats or oils, gums and glycols.

The composition may be in the form of a liquid or a solid. Liquid carriers typically comprise solutions or suspensions of water. Solid carriers are in forms known in the art, such as, for example a powder, prill, pellet, or paste, or are granular.

In one embodiment, the compositions useful in this invention do not include a lignosulfate compound. Similarly, the methods for treating parasitic worms do not include administering a lignosulfate compound.

### Yeast Cells

The intracellular components of any yeast cell capable of treating parasitic worms may be used in the compositions and methods of the invention.

For example, the yeast cells may be from the genera Aciculoconidium, Agaricomycotina, Ascomycota, Basidiomycota, Botryoascus, Brettanomyces, Bullera, Candida, Citeromyces, Clavispora, Cryptococcus, Cystofilobasium, Debaromyces, Dioszegia, Dipodascopsis, Endomyces, Entorrhizomycetes, Erythrobasidium, Fellomyces, Filobasidium, Geotrichum, Guilliermondella, Hanseniaspora, Hansenula, Hasegawaea, Hyphopichia, Incertae sedis, Issatchenkia, Kloeckera, Kluyveromyces, Leucosporidium, Lipomyces, Lodderomyces, Malassezia, Mastigomyces, Metschinikowia, Mrakia, Mrakiella, Nadsonia, Octosporomyces, Oosporidium, Pachytrichospora, Pachysolen, Penicillium, Pezizomomycotina, Phaffia, Pichia, Pityrospodium, Procandida, Prototheca, Pucciniomycotina, Rhodsporidium, Rhodotorula, Rhodotorula, Saccharomycotina, Saccharomyces, Saccharomycodes, Saccharomycopsis, Schizosaccharomycetes, Schizoblastosporion, Schwanniomyces, Selenotila, Sirobasidium, Sporidiobolus, Sporobolomyces, Stephanoascus, Sterigmatomyces, Sympodiomycopsis, Syringospora, Tibicos, Torulaspora, Taphrinomycotina, Torulopsis, Tremelloid, Trichosporon, Trigonopsis, Udeniomyces, Ustilaginomycotina, Wallemiomycetes, Waltomyces, Wickerhamia, Williopsis, Wingea, Xanthophyllomyces, Yarrowia, Zygofabospora, Zygolipomyces, Zygosaccharomyces, or any combination thereof. Preferably, the yeast cells are Saccharomyces cerevisiae, Kluyveromyces marxianus, or a combination thereof.

### Bacteria Cells

Any whole bacterial cell or lysed bacterial cell that is capable of treating parasitic worm infections in a mammal may be used in compositions and methods of the invention. By “lysed bacteria cell” it is meant that the intracellular components of the bacterial cell are used. As will be discussed below, to access the intracellular components of the bacteria cell, the bacteria may be lysed. Whole bacteria cells are also effectively used in the compositions and methods of the invention.

For example, the bacteria cells may be from the genera Acetobacterium, Acetogenium, Achromatium, Acidomonas, Acinetobacter, Acitinobacillus, Actinomyces, Actinoplanes, Aerococcus, Aeromonas, Agrobacterium, Agromonas, Agromyces, Alcaligenes, Alteromonas, Amphibacillus, Amoebobacter, Aminobacter, Anabaena, Aquaspirillum, Arthrobacter, Azomonas, Azorhizobium, Azospirillum, Azotobacter, Bacillus, Lactobacillus, Brevibacillus, Sulfobacillus, Thermobacillus, Thiobacillus, Paenibacillus, Virgibacillus, Amphibacillus, Halobacillus, Heliobacillus, Bacteroides, Beijerinckia, Bifidiobacterium, Bordetella, Bradyrhizobium, Brucella, Burkholderia, Cellulomonas, Centipeda, Chromatium, Chromobacterium, Caulobacter, Citrobacter, Chlorobium, Chloroflexus, Chloronema, Chromohalobacter, Chryseomonas, Clavibacter, Clostridium, Coprococcus, Corynebacterium, Cupriavidus, Curtobacterium, Cyanobacterium, Deinobacter, Deinococcus, Deleya, Dermocarpa, Dermocarpella, Derxia, Desulfonema, Desulfotomaculum, Desulfobulbus, Desulfomicrobium, Desulfomonas, Desulfovibrio, Thermodesulfobacterium, Desulfobacter, Desulfobacterium, Desulfococcus, Desulfomonile, Desulfonema, Desulfosarcina, Desulfurella, Desulfuromonas, Ensifer, Enterobacter, Enterococcus, Erwinia, Erythrobacter, Fibrobacter, Flavimonas, Flavobacterium, Flexibacter, Frankia, Francisella, Frateuria, Fusobacterium, Gardenerella, Gemella, Gloeobacter, Gloeocapsa, Gloeothece, Gluconobacter, Halomonas, Haemophilus, Heliobacterium, Hydrogenophaga, Kingella, Klebsiella, Kluyvera, Lactococcus, Lampropedia, Leuconostoc, Legionella, Listeria, Lysobacter, Malonomas, Marinobacter, Marinococcus, Marinomonas, Megamonas, Melissococcus, Mesophilobacter, Methylobacillus, Methanobacterium, Methanococcus, Methanomicrobium, Methanoplanus, Methylobacterium, Methylococcus, Methylomonas, Methylovorus, Microbispora, Microcossus, Microcystis, Moraxella, Morococcus, Neisseria, Nitrobacter, Nitrosomonas, Nitrosococcus, Nitrosospira, Nitrosolobus, Nitrosovibrio, Nitrospina, Nitrococcus, Nitrospira, Nocardia, Nocardiodes, Nodularia, Nostoc, Oceanospirillum, Ochrobacterum, Oligella, Oscillochlorosis, Paracoccus, Pasteurella, Pasteuria, Pediococcus, Pelobacter, Peptococcus, Phenylobacterium, Phyllobacterium, Photobacterium, Planococcus, Proteus, Providencia, Pseudanabaena, Pseudomonas, Psychrobacter, Rathayibacter, Renibacteria, Rhanella, Rhizobacter, Rhizobium, Rhizomonas, Rhodobacter, Rhodocyclus, Rhodomicrobium, Rhodopila, Rhodopseudomonas, Rhodospirillum, Roseobacter, Rugamonas, Ruminobacter, Ruminococcus, Saccharomonospora, Saccharopolyspora, Saccharococcus, Sarcina, Serpens, Serratia, Sinorhizobium, Sphingobacterium, Spirulina, Sporolactobacillus, Sporosarcina, Staphylococcus, Starria, Stenotrophomonas, Stomatococcus, Streptobacillus, Streptococcus, Streptomyces, Streptoverticillium, Syntrophobacter, Syntrophomonas, Tatunella, Taylorella, Thermoactinomycetes, Thermoleophilum, Thermomicrobium, Thermus, Thiocapsa, Thiocystis, Thiodictyon, Thiopedia, Thiospirillum, Thiospirillopsis, Thiothrix, Trichococcus, Trichodesmium, Variovorax, Vibrio, Volcaniella, Weeksella, Wolinella, Xanthobacter, Xanthomonas, Xenococcus, Xylella, Xylophilus, Yersinia, Yokonella, Zoogloea, Zymomonas, Zymophilus, or any combination thereof.

Preferably, the bacteria cells are from the genera Bacillus, Zymomonas, Rhizobia, Pseudomonas, Agrobacteria, Clostridium, Rhodopseudomonas, Arthrobacter, Flavobacteria, Azotobacter, Actinomyces, Streptomyces, Nitrobacter or any combination thereof. More preferably, the bacteria cells are from the species Zymomonas mobilis, Bacillus chitinosporus, Bacillus laterosporus or any combination thereof.

In another preferred embodiment, the bacteria are from the genus Pasteuria. Pasteuria may be cultured in vivo in nematodes, as is well known in the art, or in vitro by methods such as those described in U.S. Pat. No. 7,067,299.

### Amino Acids

Any amino acid and any combination of amino acids that is capable of improving the health of crop and/or non-crop plants, or that, in combination with the intracellular components of yeast cells or with whole or the intracellular components of lysed bacteria cells, is capable of treating parasitic worms in a mammal may be used in the compositions and methods of the invention.

Amino acids that are capable of improving the health of crop and/or non-crop plants are known in the art. Preferred amino acids include, for example, lysine, especially L-lysine (or its analogs); phenylalanine, especially L-Phenylalanine and DL-Phenylalanine; and combinations thereof.

### Proportions

To the extent any of the following components are included in a composition or method of the invention, the amount of yeast cells, bacteria cells, amino acids, and combinations thereof in the composition or method is an amount that is effective and/or sufficient to treat parasitic worm infections in a mammal.

For example, the minimum amount of intracellular components of yeast cells and of whole or intracellular components of bacteria cells is, by weight of the composition, about 0.001% by weight, preferably about 0.1% by weight, more preferably about 1% by weight, and most preferably about 10% by weight. The maximum amount of intracellular components of yeast cells is about 66% by weight, preferably about 55% by weight, and more preferably about 50% by weight of the composition.

The minimum amount of amino acids is, by weight of the composition, about 0.001% by weight, preferably about 0.1% by weight, more preferably about 1% by weight, and most preferably about 10% by weight. The maximum amount of amino acids is about 75% by weight, preferably about 66% by weight, and more preferably about 50% by weight of the composition.

### Numbers of Cells

The number of whole bacteria cells, and the number of bacteria or yeast cells that are lysed to provide the intracellular components of bacteria cells or yeast cells in the methods and compositions of the invention is any number of cells that is effective to treat parasitic worm infections in a mammal. The minimum number of cells is preferably about 1×10⁴, more preferably about 1×10⁶, and most preferably about 1×10⁷ microorganisms per gram of composition. The preferred maximum number of bacteria in the compositions and compositions of the invention is a number that is not less beneficial to the infected mammal than a lower number of cells.

### Intracellular Components of Microorganisms

Where so specified in this specification, the compositions contain the intracellular components of yeast cells and/or bacteria cells. The yeast cells and/or bacteria cells that are the source of the intracellular components are beneficial to plants. In this specification, microorganisms, e.g., bacteria and yeast, are considered to be beneficial to plants if the microorganisms colonize plant roots, or are closely associated with the plant rhizosphere, and in doing so, they promote plant growth and/or reduce disease or insect damage. Any non-beneficial characteristics of such microorganisms are outweighed by their beneficial characteristics.

In order to obtain such intracellular components, individual microorganism isolates are cultured under conditions known in the art (e.g., at about 35° C. for about 48 hours and 200 rpm in tryptic soy broth) to a sufficient cellular concentration, e.g., about 1×10⁵ to about 1×10¹² CFU/ml. The broth cultures are centrifuged and re-constituted in a suitable medium, such as a phosphate buffered solution (PBS) in preparation for the lysing event.

In one convenient lysing procedure, intracellular lysing begins with the slight heating of the buffered cellular solution (e.g., to a temperature of about 40° C. to about 50° C.). The cellular solution is mixed gently while a protease enzyme (e.g., neutral pH protease or papain) is added. The enzymatic digestion of the bacterial peptidoglycan outer cellular walls and the yeast outer cellular/transmembrane proteins liberates the intracellular components into solution. The digestion is typically complete in approximately 3-5 hours.

The microorganisms lysed to make the compositions of the invention preferably contain a favorable amount of protein; yeast—approximately 50% and bacteria—approximately 75%. The enrichment of the plant root rhizosphere with an abundance of proteins, amino acids, nucleotides, enzymes and other components favors a beneficial environment for plant nutrient uptake, growth and metabolism.

### Methods for Treating Parasitic Worm Infections in a Mammal

Any of the compositions described above may be used in a method for treating parasitic worm infections in a mammal. The parasitic infection is considered treated if there are at least about 10%, preferably about 25%, more preferably about 50%, most preferably at least about 75%, and optimally at least about 90% fewer parasites in the mammal following treatment.

Treating is also accomplished when the infected mammal has a reduction in symptoms related to the parasitic worms.

A reduction or elimination in the number of parasitic worms in a mammal, pre-, during, or post-treatment with compositions of the invention, can be measured by any means available. For example, testing stool samples from mammals undergoing treatment can be employed to measure and compare the number of worms, pre-, during and post-treatment. Any available test to measure the amounts of parasitic worms can be used.

The method comprises administering to the mammal a composition comprising an effective amount, as described above, of the intracellular components of lysed, beneficial, crop and non-crop rhizosphere-inhabiting yeast cells; lysed, beneficial, crop and non-crop rhizosphere-inhabiting bacteria cells; combinations of lysed, beneficial, crop and non-crop rhizosphere-inhabiting yeast and bacteria cells; and, optionally in each case, whole bacteria cells, and amino acids, sufficient to to treat parasitic worm infections in a mammal.

The methods for treating parasitic worms in a mammal are not limited to any particular mechanism of action, and several mechanisms of action are possible. For example, parasites may be killed directly.

In another example, nematode eggs may also be killed directly, or otherwise prevented from hatching. Alternatively, the nematodes' reproduction and/or growth stages may be adversely affected.

The action of the compositions and methods of the invention last up to one month, preferably up to two months, more preferably up to three months, most preferably up to six months, and optimally up to twelve months.

### Administration

The compositions are administered to the mammal by any means that is effective to achieve delivery to mammals. For example, the compositions can be administered orally by any means including a liquid, powder, pill, or troche. The compositions can be added to the mammal's feed or water. The compositions can also be delivered via a liquid or solid suppository.

### Mammals

According to the invention, “mammals” include human and non-human mammals. Non-human mammals include, for example, primates, pet animals such as dogs and cats, laboratory animals such as rats and mice, and farm animals such as horses, sheep, and cows.

### Miscellaneous Definitions

In this specification, groups of various parameters containing multiple members are described. Within a group of parameters, each member may be combined with any one or more of the other members to make sub-groups. For example, if the members of a genus are a, b, c, d, and e, additional sub-genuses specifically contemplated include any two, three, or four, or five of the members, e.g., a and c; a, d, and e; a, c, d, and e; a, b, c, d, and e etc.

In some cases, the members of a first genus of parameters, e.g., a, b, c, d, and e, may be combined with the members of a second genus of parameters, e.g., A, B, C, D, and E. Any member or sub-genus of the first genus may be combined with any member or sub-genus of the second genus to form additional genuses, i.e., b with C; a and c with B, D, and E, etc.

For example, in the present invention, a group of species of bacteria cells includes Zymomonas mobilis, Bacillus chitinosporus, Bacillus laterosporus, or any combination thereof. Accordingly, in addition to each species individually, sub-groups of bacteria cells include Zymomonas mobilis and Bacillus chitinosporus; Zymomonas mobilis and Bacillus laterosporus; and Bacillus chitinosporus and Bacillus laterosporus; in addition to the group of species Zymomonas mobilis, Bacillus chitinosporus, and Bacillus laterosporus.

A group of species of yeast cells includes Saccharomyces cerevisiae and Kluyveromyces marxianus. Any of the bacteria cells included in the group Zymomonas mobilis, Bacillus chitinosporus, Bacillus laterosporus individually; or any of the sub-groups of bacteria cells mentioned above, i.e., Zymomonas mobilis and Bacillus chitinosporus; Zymomonas mobilis and Bacillus laterosporus; Bacillus chitinosporus and Bacillus laterosporus; as well as the group of bacteria cells Zymomonas mobilis, Bacillus chitinosporus, and Bacillus laterosporus can be combined with either of the Yeast cells Saccharomyces cerevisiae or Kluyveromyces marxianus individually, or with the subgroup of yeast cells Saccharomyces cerevisiae and Kluyveromyces marxianus.

For example, bacterial cells from the group Zymomonas mobilis, Bacillus chitinosporus, and Bacillus laterosporus can be combined with the yeast cells from the species Saccharomyces cerevisiae. Similarly, bacterial cells from the sub-group Zymomonas mobilis and Bacillus laterosporus can be combined with yeast cells from the species Kluyveromyces marxianus. Also, bacterial cells from the group Zymomonas mobilis and Bacillus chitinosporus can be combined with yeast cells from the group Saccharomyces cerevisiae and Kluyveromyces marxianus. Etc.

A list of elements following the word “comprising” is inclusive or open-ended, i.e., the list may or may not include additional unrecited elements. A list following the words “consisting of is exclusive or closed ended, i.e., the list excludes any element not specified in the list.

All numbers in the specification are approximate unless indicated otherwise.

### Advantages of the Invention

Taken as a whole, the present invention provides a method used to to treat parasitic worm infections in a mammal. A distinct advantage of the invention is the possibility of producing compositions that are useful in effectively treating parasitic worm infections in a mammal. In a preferred embodiment of the invention, all of the compositions, and all of the methods that make use of such compositions, are free of substantial amounts of any and all synthetic chemical compounds, i.e., chemical compounds that are artificially produced by humans, and not found in the natural environment. In this regard, a substantial amount is an amount that is more than a trace amount, and that is sufficient to have a significant effect on pathogens in a rhizosphere or on the health of a plant that is in the rhizosphere.

In this specification, for example, the intracellular components of bacteria, yeast, and amino acids mentioned above are not considered to be synthetic chemicals. Some examples of synthetic chemicals that are preferably excluded from the compositions and methods of this invention include volatile nematocides, such as, for example, carbon disulfide, ethylene dibromide (EDB), 1,2-dibromo-3-chloropropane (DBCP), and 1,3-dichloropropene-1,2-dichloropropane (DD) as well as non-volatile nematocides such as, for example, O-ethyl-S,S-dipropyl phosphorodithioate (Ethoprop), 2-methyl-2-(methylthio)-propionaldehyde, O-(methylcarbamoyl)oxime (Aldicarb), 2,3-dihydro-2,2-dimethyl-7-benzofuranyl methylcarbamate (Carbofuran), O,O-diethyl-O-[p-(methylsulfinyl)phenyl]phosphorothioate (Fensulfothion), and ethyl 4-(methylthio)-m-tolyl isopropylphosphoramidte (Phenamiphus).

### EXAMPLES

### Example 1

### 50 Specific Examples of Useful Compositions

| Exem- | | | | | | |
|---|---|---|---|---|---|---|
| plary | | | | | | |
| composi- | Intracellular | Whole | Intracellular | | | |
| tion | Yeast | Bacteria | Bacteria | Amino Acids | Fertilizer | Carrier |
| 1. | 0.01% S. cerevisiae | 0.75% B. chitinosporus | | 0.5% L-Lysine | | Remainder |
| | | | | 0.75% L-Phenylalanine | | water |
| 2. | 6% K. marxianus | 8% Z. mobilis | | | | Clay in form |
| | 12% S. cerevisiae | | | | | of granule |
| 3. | 20% K. marxianus | 33% B. laterosporus | | 0.9% L-Lysine | | Charcoal in |
| | | | | 0.9% DL- | | form of |
| | | | | Phenylalanine | | pellet |
| 4. | 35% S. cerevisiae | | | 1% L-Lysine | | Sawdust in |
| | | | | 1% DL- | | form of prill |
| | | | | Phenylalanine | | |
| 5. | 25% S. cerevisiae | 1% Z. mobilis | | 3% L-Lysine | | Inert rock |
| | 25% K. marxianus | 1.5% B. laterosporus | | 1%-L Phenylalanine | | Zeolite |
| | | | | 1.5% DL- | | clinoptilolite |
| | | | | Phenylalanine | | in form of a |
| | | | | | | prill |
| 6. | 33% S. cerevisiae | 25% B. laterosporus | | | | Chalk in |
| | 33% K. marxianus | | | | | form of |
| | | | | | | paste |
| 7. | 21% K marxianus | 60% Z. mobilis | | 10% L-Phenylalanine | | Inert Zeolite |
| | | | | | | clinoptilolite |
| | | | | | | in form of |
| | | | | | | pellet |
| 8. | 0.001% K. marxianus | | | 15% L-Lysine | | Sawdust in |
| | 1.5% S. cerevisiae | | | 2% L-Phenylalanine | | form of |
| | | | | 2% DL- | | granule |
| | | | | Phenylalanine | | |
| 9. | 2.5% S. cerevisiae | 0.001% B. laterosporus | | 0.8% L-Lysine | | Inert rock in |
| | | 0.01% B. chitinosporus | | 0.8% DL- | | form of a |
| | | 0.1% Z. mobilis | | Phenylalanine | | granule |
| | | | | 0.8% L-Phenylalanine | | |
| 10. | 0.01% K. marxianus | 1.25% Z. mobilis | | 5.5% L-Lysine | | Paper waste |
| | | 15% B. laterosporus | | 8.5% L-Phenylalanine | | material in |
| | | | | 6% DL- | | form of |
| | | | | Phenylalanine | | granule |
| 11. | | | 0.60% B. chitinosporus | 0.065% DL- | | Remainder |
| | | | | Phenylalanine | | water |
| 12. | | | 10% Z. mobilis | | | Remainder |
| | | | | | | water |
| 13. | | | 0.003% B. laterosporus | 2% L-Lysine | | Clay in form |
| | | | | | | of granule |
| 14. | | | 12% Z. mobilis | 15% L-Phenylalanine | | |
| | | | 12% B. chitinosporus | | | |
| 15. | | | 25% B. laterosporus | 1.2% L-Lysine | | Sawdust in |
| | | | 5% Z. mobilis | 12% DL- | | form of prill |
| | | | | Phenylalanine | | |
| | | | | 6.8% L-Phenylalanine | | |
| 16. | | | 0.1% Z. mobilis | | | Chalk in |
| | | | 0.01% B. chitinosporus | | | form of |
| | | | | | | paste |
| 17. | | | 33% B. laterosporus | 33% L-Lysine | | Inert rock in |
| | | | | 20% L-Phenylalanine | | form of a |
| | | | | | | granule |
| 18. | | | 40% B. chitinosporus | 3% L-Lysine | | Paper waste |
| | | | 4% Z. mobilis | 3% L-Phenylalanine | | material in |
| | | | | 3% DL- | | form of |
| | | | | Phenylalanine | | granule |
| 19. | | | 60% B. laterosporus | 25% DL- | | Charcoal in |
| | | | 6% Z mobilis | Phenylalanine | | form of |
| | | | | | | pellet |
| 20. | | | 31% B. chitinosporus | 1% L-Lysine | | Inert Zeolite |
| | | | 0.1% Z mobilis | 10% DL- | | clinoptilolite |
| | | | 10% B. chitinosporus | Phenylalanine | | in form of |
| | | | | | | pellet |
| 21. | | | 50% Z. mobilis | 20% L-Phenylalanine | | Remainder |
| | | | | | | water |
| 22. | | | 66% B. laterosporus | 30% DL- | | Sawdust in |
| | | | | Phenylalanine | | form of prill |
| 23. | | | 0.001% Z. mobilis | | | Remainder |
| | | | 0.001% B. laterosporus | | | water |
| | | | 0.001% B. chitinosporus | | | |
| 24. | | | 2.5% B. chitinosporus | 0.001% L-Lysine | | Clay in form |
| | | | 0.05% Z. mobilis | 0.001% L-Phenylalanine | | of granule |
| 25. | | | 20% Z. mobilis | 18% L-Phenylalanine | | Charcoal in |
| | | | 40% B. laterosporus | 18% DL- | | form of |
| | | | | Phenylalanine | | pellet |
| 26. | | | 0.9% B. chitinosporus | 36% DL- | | Chalk in |
| | | | | Phenylalanine | | form of |
| | | | | 3.6% L-Lysine | | paste |
| 27. | | | 44% Z. mobilis | 8.8% L-Lysine | | Inert rock in |
| | | | 4.4% B. chitinosporus | 1.2% DL- | | form of a |
| | | | 0.44% B. laterosporus | Phenylalanine | | granule |
| 28. | | | 3.3% Z. mobilis | 0.5% L-Phenylalanine | | Sawdust in |
| | | | 3.3% B. laterosporus | 0.5% DL- | | form of prill |
| | | | | Phenylalanine | | |
| 29. | | | 53% B. chitinosporus | 17% L-Lysine | | Paper waste |
| | | | 7% Z. mobilis | 3% DL- | | material in |
| | | | | Phenylalanine | | form of |
| | | | | | | granule |
| 30. | | | 22% Z. mobilis | 1.5% L-Lysine | | Clay in form |
| | | | 22% B. laterosporus | 1.5% DL- | | of granule |
| | | | 22% B. chitinosporus | Phenylalanine | | |
| | | | | 1.5% L-Phenylalanine | | |
| 31. | 0.05% S. cerevisiae | | 3% B. laterosporus | 5% DL- | | Remainder |
| | | | 6% Z. mobilis | phenylalanine | | water |
| 32. | 2.5% K. marxianus | | 2.5% B. chitinosporus | | | Chalk in |
| | | | | | | form of |
| | | | | | | paste |
| 33. | 10% S. cerevisiae | | 10% Z. mobilis | 10% L-Lysine | | Clay in form |
| | 10% K. marxianus | | 10% B. chitinosporus | 5% DL- | | of granule |
| | | | 10% B. laterosporus | Phenylalanine | | |
| | | | | 5% L-Phenylalanine | | |
| 34. | 25% S. cerevisiae | | 0.001% Z. mobilis | 15% L-Lysine | | Inert Zeolite |
| | | | 1% B. chitinosporus | 15% DL- | | clinoptilolite |
| | | | 10% B. laterosporus | Phenylalanine | | in form of |
| | | | | 15% L-Phenylalanine | | prill |
| 35. | 45% K. marxianus | | 20% B. laterosporus | 0.001% L-Phenylalanine | | Inert rock in |
| | | | 5% Z. mobilis | 0.001% DL- | | form of a |
| | | | | Phenylalanine | | granule |
| 36. | 60% S. cerevisiae | | 6% B. chitinosporus | 12% L-Lysine | | Sawdust in |
| | | | | 3% L-Phenylalanine | | form of prill |
| | | | | 3% DL- | | |
| | | | | Phenylalanine | | |
| 37. | 0.001% S. cerevisiae | | 0.1% B. chitinosporus | | | magnesium |
| | 0.001% K. marxianus | | 1% Z. mobilis | | | chloride in |
| | | | 5% B. laterosporus | | | form of |
| | | | | | | granule |
| 38. | 30% S. cerevisiae | | 25% Z. mobilis | 1% L-Phenylalanine | | Paper waste |
| | 10% K. marxianus | | | 1.5% DL- | | material in |
| | | | | Phenylalanine | | form of |
| | | | | | | granule |
| 39. | 7% K. marxianus | | 0.0075% B. chitinosporus | 25% L-Lysine | | Charcoal in |
| | 18% S. cerevisiae | | 1.75% Z. mobilis | 25% L-Phenylalanine | | form of |
| | | | | | | pellet |
| 40. | 33% S. cerevisiae | | 9% Z. mobilis | 6% L-Phenylalanine | | Remainder |
| | 33% K. marxianus | | 9% B. laterosporus | | | in water |
| | | | 9% B. chitinosporus | | | |
| 41. | 10% S. cerevisiae | | | | Remainder | Prill |
| | | | | | Urea | |
| 42. | | | 3% B. laterosporus | | 18% Urea- | Remainder |
| | | | 6% Z. mobilis | | formaldehyde | water |
| | | | | | reaction | |
| | | | | | product | |
| 43. | 1% K. marxianus | | 10% B. laterosporus | | 75% ammonium | Remainder |
| | | | | | phosphate | water |
| 44. | 15% S. cerevisiae | | 25% Z. mobilis | 25% L-Lysine | 5% Urea, | Remainder |
| | | | | | 3% Triple- | water |
| | | | | | superphosphate, | |
| | | | | | 6% Potassium | |
| | | | | | nitrate | |
| 45. | 25% S. cerevisiae | | 2.5% B. laterosporus | 1% L-Lysine | Remainder | Granular |
| | 5% K. marxianus | | 2.5% Z. mobilis | 1% L-Phenylalanine | Composted | |
| | | | | 1% DL- | Manure | |
| | | | | Phenylalanine | | |
| 46. | 33% K. marxianus | | 22% Z. mobilis | 15% L-Phenylalanine | 15% Fish | Remainder |
| | | | | 15% DL- | Fertilizer | water |
| | | | | Phenylalanine | | |
| 47. | 50% S. cerevisiae | | 5% Z. mobilis | | Remainder Food | Pellet |
| | | | 5% B. laterosporus | | protein | |
| | | | 5% B. chitinosporus | | | |
| 48. | 18% S. cerevisiae | | 9% B. chitinosporus | 0.01% L-Lysine | Remainder | Granular |
| | | | 4.5% B. laterosporus | 0.05% L-Phenylalanine | Animal Cell | |
| | | | | | protein | |
| 49. | 0.05% K. marxianus | | 1.5% Z. mobilis | 33% L-Phenylalanine | 7% Methylene | Remainder |
| | 0.1% S. cerevisiae | | 0.01% B. chitinosporus | 3% L-Lysine | Urea, 5% Triple | water |
| | | | | | Superphosphate, | |
| | | | | | 5% Potassium | |
| | | | | | sulfate | |
| 50. | 40% S. cerevisiae | | 21% Z. mobilis | | 10% Ammonium | Remainder |
| | | | | | Sulfate, | water |
| | | | | | 8% Mono- | |
| | | | | | Superphosphate, | |
| | | | | | 15% Potassium | |
| | | | | | nitrate | |

### Example 2

### Lysis of Saccharomyces cerevisiae

Saccharomyces cerevisiae is cultured at 35° C. for 48 hours and 200 rpm in tryptic soy broth to a cellular concentration of 1×10⁹ CFU/ml. The broth cultures are centrifuged at 20,000 rpm for 1 hour and re-constituted in 1000 ml of phosphate buffered solution (PBS) in preparation for the lysing event. Intracellular lysing begins with the slight heating of the buffered cellular solution to a temperature of 40° C.-50° C. The cellular solution is mixed gently while a protease enzyme (e.g., neutral pH protease or papain) is added at 0.01-0.1%/wt. The enzymatic digestion of the bacterial peptidoglycan outer cellular walls and the yeasts outer cellular/transmembrane proteins liberates all intracellular components into solution and takes approximately 3-5 hours for proteolytic digestion to be complete.

### Example 3

### Lysis of Zymomonas mobilis

Zymomonas mobilis, is cultured according to the conditions of example 2, with similar results.

### Example 4

### Lysis of Bacillus chitinosporus

Bacillus chitinosporus is cultured according to the conditions of example 2, with similar results.

### Example 5

### Lysis of Bacillus laterosporus

Bacillus laterosporus is cultured according to the conditions of example 2, with similar results.

### Example 6

### The effects of LD 50 Dose Reducing Concentrations of Formula 1 on the Development of Haemonchus contortus and Trichostrongylus Eggs of Infected Sheep through an In Vitro Egg Hatch Assay and an In Vitro Larval Development Assay

### Objectives

To determine the effects of LD 50 dose reducing concentrations of Formula 1 (composition containing lysed yeast cells, lysed bacteria cells and amino acids) on the development of Haemonchus contortus and Trichostrongylus eggs of infected sheep through an in vitro egg hatch assay.

To determine the effects of LD 50 dose reducing concentrations of Formula 1 on the development of Haemonchus contortus and Trichostrongylus eggs of infected sheep through an in vitro larval development assay.

### Methods

Isolated nematode eggs from fresh feces using the lab SOP for DrenchRite larval development assay (wash through sieves of decreasing mesh size followed by sucrose gradient separation) were rinsed with deionized water and placed in a clean tube.

The average number of eggs present in two 20 μl aliquots was calculated and multiplied by a factor based on the total sample volume to determine the total number of eggs present in the sample. The volume was adjusted by either adding or removing deionized water to give an egg concentration of approximately 3500 eggs per ml. Amphotericin B antifungal solution was added at a concentration of 25 μl antifungal solution to 975 μl egg solution (final conc. in egg suspension=0.025 mg/ml−this gets diluted again 1:10 in the volume of the well for a final conc. in each well of 2.5 μg/ml).

Formula 1 was dissolved in DMSO, and doubling dilutions were prepared in DMSO. Between 8 and 11 different concentrations were used depending upon the needs of the assay. 10 μl of each concentration or 10 μl of DMSO alone (control) were then added to wells of a 96 well plate in duplicate. 150 μl of melted agar were added to all the drug wells and allowed to solidify.

20 μl of egg solution containing the antifungal were added to the wells. The plate was wrapped with Parafilm to prevent drying and incubated at 25° C. overnight. After 24 hours the plate was observed to check for hatching of the eggs. The eggs were at least 80% hatched.

After 24 hours, 20 μl of nutritive media solution (diluted 1:2) was added to the developing larvae. The plate was wrapped in Parafilm and incubated at 25° C. for 6 additional days. The plate was checked every few days and 20 μl of deionized water was added to the wells if drying occurred (a thin film of water over the agar was desired).

Since an egg hatch assay (EHA) was also being done, at 36-48 hours the plate was counted to determine the hatch rate in each well. Afterward, the plate was returned to the incubator to complete the LDA.

20 μl of 50% Lugol's solution was added to each well to terminate the assay (kills and stains the larvae). The larvae were transferred to a blank 96 well plate by washing with 50 μl of deionized water. Counting can be done immediately or the plate may be placed in the refrigerator for counting at a later time.

Both the Critical well (well observed to contain the LC50 concentration based on actual numbers) and the well containing the highest concentration of Formula 1 in which any L3 larvae appear (discriminating dose) were recorded.

Data was entered into an Excel spreadsheet, and LC50 was calculated using a logistic regression model (logit software or Graph Pad Prism).

TABLE 1
Trial Results

| Nematode Helminth | Target Animal | Efficacy (LD50) |
|---|---|---|
| Haemonchus contortus | Small Ruminant (Goat, | 299 ppm |
| | Sheep) | |
| Trichostrongylus | Small Ruminant (Goat, | 248 ppm |
| colubriformis | Sheep) | |

What is claimed is:

1. A composition for treating parasitic worm infections in a mammal, the composition comprising:
(a) an amount of intracellular components of lysed, soil-inhabiting yeast cells that are beneficial to plants, wherein the amount is sufficient to treat parasitic worm infections in the mammal; and
(b) an amount of whole or lysed bacteria cells, and a pharmaceutically suitable carrier, wherein the bacteria cells are from soil inhabiting bacteria that are beneficial to plants, and wherein the amount is sufficient to treat parasitic worm infections in the mammal.

2. The composition according to claim 1, wherein the yeast cells are from the genera Aciculoconidium, Agaricomycotina, Ascomycota, Basidiomycota, Botryoascus, Brettanomyces, Bullera, Candida, Citeromyces, Clavispora, Cryptococcus, Cystofilobasium, Debaromyces, Dioszegia, Dipodascopsis, Endomyces, Entorrhizomycetes, Erythrobasidium, Fellomyces, Filobasidium, Geotrichum, Guilliermondella, Hanseniaspora, Hansenula, Hasegawaea, Hyphopichia, Incertae sedis, Issatchenkia, Kloeckera, Kluyveromyces, Leucosporidium, Lipomyces, Lodderomyces, Malassezia, Mastigomyces, Metschinikowia, Mrakia, Mrakiella, Nadsonia, Octosporomyces, Oosporidium, Pachytrichospora, Pachysolen, Penicillium, Pezizomomycotina, Phaffia, Pichia, Pityrospodium, Procandida, Prototheca, Pucciniomycotina, Rhodsporidium, Rhodotorula, Rhodotorula, Saccharomycotina, Saccharomyces, Saccharomycodes, Saccharomycopsis, Schizosaccharomycetes, Schizoblastosporion, Schwanniomyces, Selenotila, Sirobasidium, Sporidiobolus, Sporobolomyces, Stephanoascus, Sterigmatomyces, Sympodiomycopsis, Syringospora, Tibicos, Torulaspora, Taphrinomycotina, Torulopsis, Tremelloid, Trichosporon, Trigonopsis, Udeniomyces, Ustilaginomycotina, Wallemiomycetes, Waltomyces, Wickerhamia, Williopsis, Wingea, Xanthophyllomyces, Yarrowia, Zygofabospora, Zygolipomyces, Zygosaccharomyces, or any combination thereof.

3. The composition according to claim 1, wherein the yeast cells are Saccharomyces cerevisiae, Kluyveromyces marxianus, or any combination thereof.

4. The composition according to claim 1, wherein the minimum amount of intracellular components of yeast cells is about 0.001% by weight of the composition and the maximum amount of intracellular components of yeast cells is about 20% by weight of the composition.

5. The composition according to claim 1, wherein the bacteria cells are from the genera Acetobacterium, Acetogenium, Achromatium, Acidomonas, Acinetobacter, Acitinobacillus, Actinomyces, Actinoplanes, Aerococcus, Aeromonas, Agrobacterium, Agromonas, Agromyces, Alcaligenes, Alteromonas, Amphibacillus, Amoebobacter, Aminobacter, Anabaena, Aquaspirillum, Arthrobacter, Azomonas, Azorhizobium, Azospirillum, Azotobacter, Bacillus, Lactobacillus, Brevibacillus, Sulfobacillus, Thermobacillus, Thiobacillus, Paenibacillus, Virgibacillus, Amphibacillus, Halobacillus, Heliobacillus, Bacteroides, Beijerinckia, Bifidiobacterium, Bordetella, Bradyrhizobium, Brucella, Burkholderia, Cellulomonas, Centipeda, Chromatium, Chromobacterium, Caulobacter, Citrobacter, Chlorobium, Chloroflexus, Chloronema, Chromohalobacter, Chryseomonas, Clavibacter, Clostridium, Coprococcus, Corynebacterium, Cupriavidus, Curtobacterium, Cyanobacterium, Deinobacter, Deinococcus, Deleya, Dermocarpa, Dermocarpella, Derxia, Desulfonema, Desulfotomaculum, Desulfobulbus, Desulfomicrobium, Desulfomonas, Desulfovibrio, Thermodesulfobacterium, Desulfobacter, Desulfobacterium, Desulfococcus, Desulfomonile, Desulfonema, Desulfosarcina, Desulfurella, Desulfuromonas, Ensifer, Enterobacter, Enterococcus, Erwinia, Erythrobacter, Fibrobacter, Flavimonas, Flavobacterium, Flexibacter, Frankia, Francisella, Frateuria, Fusobacterium, Gardenerella, Gemella, Gloeobacter, Gloeocapsa, Gloeothece, Gluconobacter, Halomonas, Haemophilus, Heliobacterium, Hydrogenophaga, Kingella, Klebsiella, Kluyvera, Lactococcus, Lampropedia, Leuconostoc, Legionella, Listeria, Lysobacter, Malonomas, Marinobacter, Marinococcus, Marinomonas, Megamonas, Melissococcus, Mesophilobacter, Methylobacillus, Methanobacterium, Methanococcus, Methanomicrobium, Methanoplanus, Methylobacterium, Methylococcus, Methylomonas, Methylovorus, Microbispora, Microcossus, Microcystis, Moraxella, Morococcus, Neisseria, Nitrobacter, Nitrosomonas, Nitrosococcus, Nitrosospira, Nitrosolobus, Nitrosovibrio, Nitrospina, Nitrococcus, Nitrospira, Nocardia, Nocardiodes, Nodularia, Nostoc, Oceanospirillum, Ochrobacterum, Oligella, Oscillochlorosis, Paracoccus, Pasteurella, Pasteuria, Pediococcus, Pelobacter, Peptococcus, Phenylobacterium, Phyllobacterium, Photobacterium, Planococcus, Proteus, Providencia, Pseudanabaena, Pseudomonas, Psychrobacter, Rathayibacter, Renibacteria, Rhanella, Rhizobacter, Rhizobium, Rhizomonas, Rhodobacter, Rhodocyclus, Rhodomicrobium, Rhodopila, Rhodopseudomonas, Rhodospirillum, Roseobacter, Rugamonas, Ruminobacter, Ruminococcus, Saccharomonospora, Saccharopolyspora, Saccharococcus, Sarcina, Serpens, Serratia, Sinorhizobium, Sphingobacterium, Spirulina, Sporolactobacillus, Sporosarcina, Staphylococcus, Starria, Stenotrophomonas, Stomatococcus, Streptobacillus, Streptococcus, Streptomyces, Streptoverticillium, Syntrophobacter, Syntrophomonas, Tatunella, Taylorella, Thermoactinomycetes, Thermoleophilum, Thermomicrobium, Thermus, Thiocapsa, Thiocystis, Thiodictyon, Thiopedia, Thiospirillum, Thiospirillopsis, Thiothrix, Trichococcus, Trichodesmium, Variovorax, Vibrio, Volcaniella, Weeksella, Wolinella, Xanthobacter, Xanthomonas, Xenococcus, Xylella, Xylophilus, Yersinia, Yokonella, Zoogloea, Zymomonas, Zymophilus, or any combination thereof.

6. The composition according to claim 1, wherein bacteria cells are from the genera Bacillus, Zymomonas, Rhizobia, Pseudomonas, Agrobacteria, Clostridium, Rhodopseudomonas, Arthrobacter, Flavobacteria, Azotobacter, Actinomyces, Streptomyces, Nitrobacter, or any combination thereof.

7. The composition according to claim 1, wherein bacteria cells are from the species Zymomonas mobilis, Bacillus chitinosporus, Bacillus laterosporus, or any combination thereof.

8. The composition according to claim 1, wherein the minimum amount of intracellular components of bacteria cells is about 0.001% by weight of the composition and the maximum amount of intracellular components of bacteria cells is about 20% by weight of the composition.

9. The composition according to claim 1, wherein the composition is a liquid

10. The composition according to claim 1, wherein the composition is a solid.

11. The composition according to claim 10, wherein the composition is in the form of a powder, pill or suppository.

12. The composition according to claim 11, wherein the parasitic worm is selected from the group consisting of cestodes, nematodes and trematodes, or any combination thereof.

13. The composition according to claim 11, wherein the composition does not include a lignosulfonate compound.

14. The composition according to claim 81, wherein the composition further comprises an amount of amino acids sufficient to treat parasitic worm infections in the mammal.

15. The composition according to claim 14, wherein the amino acids comprise lysine, phenylalanine, or any combination thereof.

16. The composition according to claim 14, wherein at least one of the amino acids is L-lysine.

17. The composition according to claim 14, wherein at least one of the amino acids is phenylalanine and the phenylalanine is L-phenylalanine, DL-phenylalanine, or any combination thereof.

18. The composition according to claim 14, wherein the minimum amount of amino acids is about 0.001% by weight of the composition and the maximum amount of amino acids is about 25% by weight of the composition.

19. The composition according to claim 1, wherein the composition contains no synthetic chemical compounds.

20. The composition according to claim 1, wherein the composition further comprises a pharmaceutically suitable carrier.

21. A method for treating parasitic worm infections in a mammal, the method comprising administering to the mammal a composition comprising:
(a) an amount of intracellular components of lysed, soil-inhabiting yeast cells that are beneficial to plants, wherein the amount is sufficient to treat parasitic worm infections in the mammal; and
(b) an amount of whole or lysed bacteria cells, and a pharmaceutically suitable carrier, wherein the bacteria cells are from soil inhabiting bacteria that are beneficial to plants, and wherein the amount is sufficient to treat parasitic worm infections in the mammal.

22. The method according to claim 21, wherein the parasitic worm is selected from the group consisting of cestodes, nematodes and trematodes, or any combination thereof.

23. The method according to claim 21, wherein the yeast cells are from the genera Aciculoconidium, Agaricomycotina, Ascomycota, Basidiomycota, Botryoascus, Brettanomyces, Bullera, Candida, Citeromyces, Clavispora, Cryptococcus, Cystofilobasium, Debaromyces, Dioszegia, Dipodascopsis, Endomyces, Entorrhizomycetes, Erythrobasidium, Fellomyces, Filobasidium, Geotrichum, Guilliermondella, Hanseniaspora, Hansenula, Hasegawaea, Hyphopichia, Incertae sedis, Issatchenkia, Kloeckera, Kluyveromyces, Leucosporidium, Lipomyces, Lodderomyces, Malassezia, Mastigomyces, Metschinikowia, Mrakia, Mrakiella, Nadsonia, Octosporomyces, Oosporidium, Pachytrichospora, Pachysolen, Penicillium, Pezizomomycotina, Phaffia, Pichia, Pityrospodium, Procandida, Prototheca, Pucciniomycotina, Rhodsporidium, Rhodotorula, Rhodotorula, Saccharomycotina, Saccharomyces, Saccharomycodes, Saccharomycopsis, Schizosaccharomycetes, Schizoblastosporion, Schwanniomyces, Selenotila, Sirobasidium, Sporidiobolus, Sporobolomyces, Stephanoascus, Sterigmatomyces, Sympodiomycopsis, Syringospora, Tibicos, Torulaspora, Taphrinomycotina, Torulopsis, Tremelloid, Trichosporon, Trigonopsis, Udeniomyces, Ustilaginomycotina, Wallemiomycetes, Waltomyces, Wickerhamia, Williopsis, Wingea, Xanthophyllomyces, Yarrowia, Zygofabospora, Zygolipomyces, Zygosaccharomyces, or any combination thereof.

24. The method according to claim 21, wherein the yeast cells are Saccharomyces cerevisiae, Kluyveryomyces marxianus, or a combination thereof.

25. The method according to claim 21, wherein the minimum amount of intracellular components of yeast cells is about 0.001% by weight of the composition and the maximum amount of intracellular components of yeast cells is about 20% by weight of the composition.

26. The method according to claim 21, wherein the bacteria cells are from the genera Acetobacterium, Acetogenium, Achromatium, Acidomonas, Acinetobacter, Acitinobacillus, Actinomyces, Actinoplanes, Aerococcus, Aeromonas, Agrobacterium, Agromonas, Agromyces, Alcaligenes, Alteromonas, Amphibacillus, Amoebobacter, Aminobacter, Anabaena, Aquaspirillum, Arthrobacter, Azomonas, Azorhizobium, Azospirillum, Azotobacter, Bacillus, Lactobacillus, Brevibacillus, Sulfobacillus, Thermobacillus, Thiobacillus, Paenibacillus, Virgibacillus, Amphibacillus, Halobacillus, Heliobacillus, Bacteroides, Beijerinckia, Bifidiobacterium, Bordetella, Bradyrhizobium, Brucella, Burkholderia, Cellulomonas, Centipeda, Chromatium, Chromobacterium, Caulobacter, Citrobacter, Chlorobium, Chloroflexus, Chloronema, Chromohalobacter, Chryseomonas, Clavibacter, Clostridium, Coprococcus, Corynebacterium, Cupriavidus, Curtobacterium, Cyanobacterium, Deinobacter, Deinococcus, Deleya, Dermocarpa, Dermocarpella, Derxia, Desulfonema, Desulfotomaculum, Desulfobulbus, Desulfomicrobium, Desulfomonas, Desulfovibrio, Thermodesulfobacterium, Desulfobacter, Desulfobacterium, Desulfococcus, Desulfomonile, Desulfonema, Desulfosarcina, Desulfurella, Desulfuromonas, Ensifer, Enterobacter, Enterococcus, Erwinia, Erythrobacter, Fibrobacter, Flavimonas, Flavobacterium, Flexibacter, Frankia, Francisella, Frateuria, Fusobacterium, Gardenerella, Gemella, Gloeobacter, Gloeocapsa, Gloeothece, Gluconobacter, Halomonas, Haemophilus, Heliobacterium, Hydrogenophaga, Kingella, Klebsiella, Kluyvera, Lactococcus, Lampropedia, Leuconostoc, Legionella, Listeria, Lysobacter, Malonomas, Marinobacter, Marinococcus, Marinomonas, Megamonas, Melissococcus, Mesophilobacter, Methylobacillus, Methanobacterium, Methanococcus, Methanomicrobium, Methanoplanus, Methylobacterium, Methylococcus, Methylomonas, Methylovorus, Microbispora, Microcossus, Microcystis, Moraxella, Morococcus, Neisseria, Nitrobacter, Nitrosomonas, Nitrosococcus, Nitrosospira, Nitrosolobus, Nitrosovibrio, Nitrospina, Nitrococcus, Nitrospira, Nocardia, Nocardiodes, Nodularia, Nostoc, Oceanospirillum, Ochrobacterum, Oligella, Oscillochlorosis, Paracoccus, Pasteurella, Pasteuria, Pediococcus, Pelobacter, Peptococcus, Phenylobacterium, Phyllobacterium, Photobacterium, Planococcus, Proteus, Providencia, Pseudanabaena, Pseudomonas, Psychrobacter, Rathayibacter, Renibacteria, Rhanella, Rhizobacter, Rhizobium, Rhizomonas, Rhodobacter, Rhodocyclus, Rhodomicrobium, Rhodopila, Rhodopseudomonas, Rhodospirillum, Roseobacter, Rugamonas, Ruminobacter, Ruminococcus, Saccharomonospora, Saccharopolyspora, Saccharococcus, Sarcina, Serpens, Serratia, Sinorhizobium, Sphingobacterium, Spirulina, Sporolactobacillus, Sporosarcina, Staphylococcus, Starria, Stenotrophomonas, Stomatococcus, Streptobacillus, Streptococcus, Streptomyces, Streptoverticillium, Syntrophobacter, Syntrophomonas, Tatunella, Taylorella, Thermoactinomycetes, Thermoleophilum, Thermomicrobium, Thermus, Thiocapsa, Thiocystis, Thiodictyon, Thiopedia, Thiospirillum, Thiospirillopsis, Thiothrix, Trichococcus, Trichodesmium, Variovorax, Vibrio, Volcaniella, Weeksella, Wolinella, Xanthobacter, Xanthomonas, Xenococcus, Xylella, Xylophilus, Yersinia, Yokonella, Zoogloea, Zymomonas, Zymophilus, or any combination thereof.

27. The method according to claim 21, wherein the bacteria cells are from the genera Bacillus, Zymomonas, Rhizobia, Pseudomonas, Agrobacteria, Clostridium, Rhodopseudomonas, Arthrobacter, Flavobacteria, Azotobacter, Actinomyces, Streptomyces, Nitrobacter, or any combination thereof.

28. The method according to claim 21, wherein the bacteria cells are from the species Zymomonas mobilis, Bacillus chitinosporus, Bacillus laterosporus, or any combination thereof.

29. The method according to claim 21, wherein the minimum amount of intracellular components of bacteria cells is about 0.001% by weight of the composition and the maximum amount of intracellular components of bacteria cells is about 20% by weight of the composition.

30. The method according to claim 21, wherein the composition is a liquid.

31. The method according to claim 21, wherein the composition is a solid.

32. The method according to claim 21, wherein the composition is in the form of a powder, pill or suppository.

33. The method according to claim 21, wherein the method does not include administering a lignosulfonate compound to the mammal.

34. The method according to claim 21, wherein the composition further comprises an amount of amino acids sufficient to treat parasitic worm infections in the mammal.

35. The method according to claim 34, wherein the minimum amount of amino acids is about 0.001% by weight of the composition and the maximum amount of amino acids is about 25% by weight of the composition.

36. The method according to claim 34, wherein the amino acids comprise lysine, phenylalanine, or any combination thereof.

37. The method according to claim 34, wherein at least one of the amino acids is L-lysine.

38. The method according to claim 34, wherein at least one of the amino acids is phenylalanine and the phenylalanine is L-phenylalanine, DL-phenylalanine, or any combination thereof.

39. The method according to claim 21, wherein the composition contains no synthetic chemical compounds.

40. The method according to claim 21, wherein the composition further comprises a pharmaceutically suitable carrier.
